(19) United States
(12) Patent Application Publication
(10) Pub. No.: US 2024/0417760 A1
(43) Pub. Date: Dec. 19, 2024

(54) METHOD TO PRODUCE ENANTIOMERS OF UNDECAVERTOL

(71) Applicant: GIVAUDAN SA, VERNIER (CH)

(72) Inventors: Marc BIERMANN, Zürich (CH); Corinne BAUMGARTNER, Fällanden (CH); Simon ELLWOOD, Kent (GB)

(21) Appl. No.: 18/815,248

(22) Filed: Aug. 26, 2024

Related U.S. Application Data
(63) Continuation of application No. 16/964,772, filed on Jul. 24, 2020.

(30) Foreign Application Priority Data
Feb. 14, 2018 (GB) 1802383.8

(51) Int. Cl. C12P 7/04; C12N 9/04; C12P 41/00

(52) U.S. Cl. CPC C12P 7/04; C12N 9/0006; C12P 41/002; C12Y 101/01001; C12Y 101/01002; C12Y 101/01071

(57) ABSTRACT

A method for increasing the proportion of an enantiomer of undecavertol in an enantiomeric mixture of undecavertol, a method for stereoselectively synthesising undecavertol, and the products thereof.

### CROSS REFERENCE TO RELATED APPLICATIONS

This application is a continuation of U.S. Ser. No. 16/964,772, filed 24 Jul. 2020, which is a national stage application of International Application No. PCT/EP2019/053498, filed 13 Feb. 2019, which claims priority from Great Britain Patent Application No. 1802383.8, filed 14 Feb. 2018, both of which applications are incorporated herein by reference in their entireties.

### TECHNICAL FIELD

The present invention relates generally to a method for increasing the proportion of an enantiomer of undecavertol in an enantiomeric mixture of undecavertol, in particular a method for increasing the proportion of (R)-enantiomer of undecavertol in an enantiomeric mixture. The present invention also relates generally to a method of stereoselectively synthesising undecavertol from undecavertone. The present invention further relates to the enantiomeric mixtures formed by these methods and the use of these enantiomeric mixtures as a fragrance.

### BACKGROUND

Undecavertol (chemical name 4-methyl-3-decen-5-ol) is a molecule having a floral, green, fresh, violet leaves odour and is well known as fragrance ingredient. The different enantiomers of undecavertol have different odour strengths, the (S)-enantiomer being the weaker enantiomer, and therefore different enantiomers may be selected depending on the composition into which the undecavertol is to be incorporated. One method to selectively obtain (R)-undecavertol was published by Brenna et al., “Bio-catalysed synthesis of optically active Undecavertol enantiomers”, Tetrahedron: Asymmetry, 16 (2005), pages 1997-1999. This method uses a lipase to selectively acetylate (R)-undecavertol from a racemic mixture of undecavertol with vinyl acetate over 6 days in refluxing organic solvent. Unreacted (S)-undecavertol and acetylated (R)-undecavertol are then separated via column chromatography, yielding only 23% pure acetylated (R)-undecavertol. The acetylated (R)-undecavertol is then hydrolysed to obtain (R)-undecavertol with an 89% yield (21% yield overall). It is therefore desirable to provide alternative and/or improved methods for enhancing the enantiomeric purity of enantiomeric mixtures of undecavertol.

### SUMMARY OF THE INVENTION

In accordance with a first aspect of the present invention there is provided a method of increasing the proportion of an enantiomer of undecavertol in an enantiomeric mixture of undecavertol, the method comprising contacting an enantiomeric mixture of undecavertol with an alcohol dehydrogenase (ADH) and an ADH-cofactor. In certain embodiments, the method is for increasing the proportion of the (R)-enantiomer of undecavertol in the enantiomeric mixture.

In accordance with a second aspect of the present invention there is provided a method of stereoselectively synthesising undecavertol from undecavertone. In certain embodiments, the method comprises contacting undecavertone with an alcohol dehydrogenase (ADH) and an ADH-cofactor. In certain embodiments, the method comprises first synthesising undecavertone from undecavertol. In certain embodiments, the undecavertone may be asymmetrically hydrogenated using a catalyst.

In accordance with a third aspect of the present invention there is provided an enantiomeric mixture of undecavertol obtained by and/or obtainable by the method of any aspect or embodiment of the present invention.

In accordance with a fourth aspect of the present invention there is provided an enantiomeric mixture of undecavertol having an enantiomeric excess equal to or greater than about 94%.

In accordance with a fifth aspect of the present invention there is provided the use of an enantiomeric mixture of undecavertol of any aspect or embodiment of the present invention as a fragrance.

In accordance with a sixth aspect of the present invention there is provided a fragrance composition comprising an enantiomeric mixture of undecavertol of any aspect or embodiment of the present invention.

Certain embodiments of any aspect of the present invention may provide one or more of the following advantages:
method can be carried out in purely aqueous (non-organic) media (e.g. when using NAD(P)H oxidase as the cofactor regeneration system), without any additional co-solvent;
green and sustainable process;
high stereoselectivity;
reaction rate rapidly decreases to almost zero after one enantiomer has almost completely reacted so close monitoring to avoid oxidation of the second enantiomer is not necessary;
enzymes have high tolerance to high concentrations (e.g. up to 80 vol %) of hydrophobic substrate;
reduced number of reaction steps;
reduced need for additional reagents;
relatively low temperature required for reaction;
shorter length of time required for reaction;
reduced waste;
improved atom economy.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based, at least in part, on the surprising finding that alcohol dehydrogenases have a high stereoselectivity to undecavertol, even though the two residues at the secondary alcohol or ketone are approximately the same size. A high stereoselectivity a person skilled in the art would in general only expect when the size of the residues are substantially different. This means that, in the reaction using undecavertol as the substrate, one enantiomer is converted to undecavertone (4-methyldec-3-en-5-one), leaving the other enantiomer almost untouched. This means that the reaction substantially stops as soon as the one enantiomer is converted. For example, starting with a racemic mixture comprising the (R)-enantiomer and (S)-enantiomer in a ratio of 1:1, the reaction may stop at approximately 50% conversion, so close monitoring of the conversion to avoid oxidation of the second enantiomer is not required.

Thus, there is provided herein a method for increasing the enantiomeric purity of undecavertol, the method comprising contacting an enantiomeric mixture of undecavertol with an alcohol dehydrogenase (ADH) and an ADH-cofactor.

There is also provided herein a method for stereoselectively synthesising undecavertol, the method comprising contacting undecavertone with an alcohol dehydrogenase (ADH) and an ADH-cofactor.

The commercially available undecavertol (4-methyl-3-decen-5-ol) is a mixture of (E)- and (Z)-isomers comprising at least 97% of the (E)-isomer, having the structure shown below. It may also be referred to as figovert, violet decenol, or kevertol.

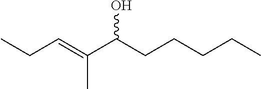

An enantiomeric mixture of undecavertol refers to a mixture of (R)-enantiomers and (S)-enantiomers of undecavertol. The enantiomeric mixture of undecavertol may be obtained by any method known to a skilled person, for example by the method described in U.S. Pat. No. 4,482,762, the contents of which are incorporated herein by reference.

An enantiomeric mixture of undecavertol is used as the substrate for the alcohol dehydrogenase catalyzed oxidation. The enantiomeric mixture of undecavertol may, for example, have a ratio of (R)-enantiomer to (S)-enantiomer ranging from about 45:55 to about 55:45 or from about 46:54 to about 54:46 or from about 47:53 to about 53:47 or from about 48:52 to about 52:48 or from about 49:51 to about 51:49. The enantiomeric mixture of undecavertol may, for example, be a racemic mixture of undecavertol, which is a mixture that contains equal amounts of the (R)-enantiomer and (S)-enantiomer (a ratio of (R)-enantiomer to (S)-enantiomer of 50:50).

The enantiomeric mixture of undecavertol used as the ADH substrate may, for example, have an enantiomeric excess equal to or less than about 10%. For example, the enantiomeric mixture of undecavertol may have an enantiomeric excess equal to or less than about 9% or equal to or less than about 8% or equal to or less than about 7% or equal to or less than about 6% or equal to or less than about 5% or equal to or less than about 4% or equal to or less than about 3% or equal to or less than about 2% or equal to or less than about 1%. For example, the enantiomeric mixture of undecavertol may have an enantiomeric excess of about 0%. The enantiomeric mixture of undecavertol may, for example, have an enantiomeric excess of the (S)-enantiomer or the (R)-enantiomer.

Enantiomeric excess (ee) is a measurement of the difference between the amounts of each enantiomer. For example, a mixture of 70% of one enantiomer and 30% of the other enantiomer has an enantiomeric excess of 40%. A racemic mixture has an enantiomeric excess of 0% and a completely pure enantiomer has an enantiomeric excess of 100%. The amount of each enantiomer in the mixture may, for example, be measured using methods such as chiral column chromatography and NMR spectroscopy.

The method provided herein may be a method for increasing the proportion of one enantiomer in an enantiomeric mixture of undecavertol compared to the other enantiomer. The method may, for example, reduce the enantiomeric excess of one enantiomer. For example, the starting enantiomeric mixture may have an excess of (S)-enantiomer and the method provided herein may increase the proportion of (R)-enantiomer to (S)-enantiomer such that the enantiomeric excess of the (S)-enantiomer is reduced, for example the method provided herein may increase the proportion of (R)-enantiomer such that the (R)-enantiomer is in excess of the (S)-enantiomer. The method may, for example, increase the enantiomeric excess of one enantiomer. For example, the starting enantiomeric mixture may have an excess of (R)-enantiomer and the method provided herein may increase the proportion of (R)-enantiomer such that the enantiomeric excess of the (R)-enantiomer is increased.

Undecavertone (4-methyldec-3-en-5-one) has the structure shown below. It may, for example, be obtained by converting undecavertol using ADH as described herein. It may, for example, be obtained by any method known to a skilled person, for example by an Oppenauer oxidation reaction as described in EP1690848.

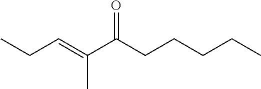

The method provided herein may be a method for stereoselectively synthesising undecavertol from undecavertone. This means that the majority of the undecavertone that is converted to undecavertol is converted to the same enantiomer of undecavertol. For example, at least about 60% or at least about 70% or at least about 75% or at least about 80% or at least about 85% or at least about 90% or at least about 92% or at least about 94% or at least about 95% or at least about 96% or at least about 97% or at least about 98% or at least about 99% of the undecavertone that is converted to undecavertol is converted to a single enantiomer of undecavertol. The reaction may, for example, be selective for the (R)-enantiomer or the (S)-enantiomer.

The method provided herein may result in an undecavertol product having an enantiomeric excess equal to or greater than about 94%. For example, the method may result in a product having an enantiomeric excess equal to or greater than about 95% or equal to or greater than about 96% or equal to or greater than about 97% or equal to or greater than about 98% or equal to or greater than about 99%. In certain embodiments, the method may result in a product having an enantiomeric excess equal to or less than about 100% or equal to or less than about 99.9% or equal to or less than about 99.8% or equal to or less than about 99.7% or equal to or less than about 99.6% or equal to or less than about 99.5%. In certain embodiments, the method may result in a product having an enantiomeric excess of 100%, in other words the product is a completely pure enantiomer. The product may comprise an enantiomeric excess of either the (R)-enantiomer or the (S)-enantiomer. In certain embodiments, the product comprises an enantiomeric excess of (R)-enantiomer.

The methods provided herein may comprise contacting the enantiomeric mixture of undecavertol with an alcohol dehydrogenase (ADH) and an ADH-cofactor. The methods provided herein may comprise contacting undecavertone with an alcohol dehydrogenase (ADH) and an ADH-cofactor.

Alcohol dehydrogenases (ADH) are a group of enzymes that facilitate the conversion of alcohols to aldehydes or ketones and the conversion of aldehydes or ketones to alcohols. If this class of enzymes is used for the conversion of aldehydes or ketones to alcohols they are more commonly referred to as ketoreductases (KRED). The ADH may be of any type suitable for carrying out a stereospecific conversion of undecavertol to undecavertone and/or undecavertone to undecavertol. The ADH may, for example, be a eukaryotic ADH, a bacterial ADH or an archaebacterial ADH, for example, a human ADH, an animal ADH, a plant ADH, a fungal ADH such as yeast ADH, or a combination of one or more thereof. The ADH may, for example, be a Class I, II, III, IV, V or VI ADH or a combination of one or more thereof. The ADH may, for example, be a long-chain ADH, a short-chain ADH or an iron-containing ADH.

Enzymes may also be defined by their function according to the EC classification. The Enzyme Commission number (EC number) is a numerical classification scheme for enzymes, based on the chemical reactions they catalyse. The ADH suitable for carrying out the stereospecific conversion described herein may, for example, belong to the EC 1.1.1 class (including the subclasses EC 1.1.1.1, EC 1.1.1.2; EC 1.1.1.54, and EC 1.1.1.71), which are oxidoreductases acting on the CH—OH groups of donors with NAD(+) or NADP(+) as acceptors.

The ADH may, for example, be one or more of the ADH enzymes used in the Examples below, for example KRED-P1-B10 obtained from Codexis, Inc., Redwood City, (USA) or ADH-87 or ADH-109 or ADH-170 or ADH-171 or ADH-172 or ADH-174 obtained from c-LEcta GmbH, Leipzig, (Germany) or GV-K-120 or GV-K-133 commercially available from EnzymeWorks, Suzhou (China), all of which are S-selective for undecavertol, which belong to the EC 1.1.1 class selectively forming the R-enantiomer of undecavertol. Or the ADH may, for example, be one or more of the following ADH enzymes PRO-258 (commercially available from Prozomix Ltd., Haltwhistle, UK), KRED-P3-G09 and KRED-P3-H12 (both commercially available from Codexis, Inc., Redwood City, USA), all of which are R-selective for undecavertol, which belong to the EC 1.1.1 class selectively forming the S-enantiomer of undecavertol.

Alcohol dehydrogenase cofactors (ADH-cofactors) are cofactors that assist the ADH enzyme during the catalysis of reactions. The ADH-cofactor used in the method provided herein may be of any type suitable for assisting the conversion of undecavertol to undecavertone and/or undecavertone to undecavertol. The cofactor may, for example, be an inorganic or organic molecule. The ADH-cofactor may, for example, be selected from nicotinamide adenine dinucleotide (NAD), nicotinamide adenine dinucleotide phosphate (NADP), quinoid cofactors, zinc or a combination thereof. Quinoid cofactors are cofactors having a structure based on quinone. The ADH-cofactor may, for example, be selected from NAD, NADP or a combination thereof. The ADH-cofactor may, for example, be zinc and one or both of NAD and NADP. The ADH-cofactor may be reduced simultaneously with the conversion of undecavertol to undecavertone. For example, the oxidised form of NAD and/or NADP (NAD⁺ and NADP⁺) may be reduced simultaneously with the conversion of the undecavertol to undecavertone to form NADH and/or NADPH respectively. The ADH-cofactor may be oxidised simultaneously with the conversion of undecavertone to undecavertol. For example, the reduced form of NAD and/or NADP (NADH and NADPH) may be oxidised simultaneously with the conversion of the undecavertone to undecavertol to form NAD⁺ and NADP⁺ respectively.

The methods provided herein may further comprise contacting the ADH-cofactor with an ADH-cofactor regeneration system. The ADH-cofactor regeneration system serves to regenerate the ADH-cofactor after it has been used to assist the conversion of undecavertol to undecavertone or the conversion of undecavertone to undecavertol. The ADH-cofactor regeneration may, for example, regenerate the oxidised form of the cofactor, for example the oxidised form of NAD and/or NADP (NAD⁺ and NADP⁺). The ADH-cofactor regeneration may, for example, regenerate the reduced form of the cofactor, for example the reduced form of NAD and/or NADP (NADH and NADPH). The ADH-cofactor regeneration used in the methods provided herein may be of any type suitable for regenerating an ADH-cofactor that is useful in the conversion of undecavertol to undecavertone and/or the conversion of undecavertone to undecavertol by ADH.

The ADH-cofactor regeneration system may, for example, be a substrate-coupled regeneration system. A substrate-coupled regeneration system provides an additional substrate for the ADH which regenerates the ADH-cofactor when it is converted by the ADH. For example, the substrate-coupled regeneration system may regenerate an oxidised or a reduced ADH-cofactor when it is converted by the ADH. No further enzymes are required. The substrate-coupled regeneration system may therefore comprise an alcohol, aldehyde or ketone as co-substrate. The substrate-coupled regeneration system may then convert the aldehyde or ketone to the corresponding alcohol or convert the alcohol to the corresponding aldehyde or ketone. For example, an oxidised ADH-cofactor may be formed when an aldehyde or ketone is converted by the ADH to its corresponding alcohol. For example, a reduced ADH-cofactor may be formed when an alcohol is converted by the ADH to its corresponding ketone or aldehyde. The substrate-coupled regeneration system may, for example, comprise acetone, for example when the regeneration system is used to regenerate the ADH-cofactor used to convert undecavertol to undecavertone. Acetone is converted to isopropanol by ADH simultaneously with the oxidation of the ADH-cofactor (e.g. oxidation of NADH and/or NADPH to NAD⁺ and/or NADP⁺). Thus, isopropanol is a by-product of the reaction. The substrate-coupled regeneration system may, for example, comprise isopropanol, for example when the regeneration system is used to regenerate the ADH-cofactor used to convert undecavertone to undecavertol. Isopropanol is converted to acetone by ADH simultaneously with the reduction of the ADH-cofactor (e.g. reduction NAD⁺ and/or NADP⁺ to NADH and/or NADPH respectively). Thus, acetone is a by-product of the reaction.

The ADH-cofactor regeneration system may, for example, be an enzyme-coupled regeneration system. An enzyme-coupled regeneration system provides an additional enzyme which regenerates the ADH-cofactor as a result of a reaction it catalyses. Any enzyme using NADH or NADPH as cofactor could be used in an ADH-cofactor regeneration system. The enzyme-coupled regeneration system may, for example, comprise a dehydrogenase, a reductase, a monooxygenase, a hydroxylase, a dioxygenase or a combination of one or more thereof. The enzyme-coupled regeneration system may, for example, comprise any other catalyst, for example any inorganic complex, that regenerates the ADH-cofactor as a result of a reaction it catalyses. The enzyme-coupled regeneration system may, for example, comprise NADH oxidase, NADPH oxidase, iron (III) porphyrine complex, laccase, lactate dehydrogenase, glutamate dehydrogenase, formiate dehydrogenase (FDH), glucose dehydrogenase (GDH), glucose-6-phosphate dehydrogenase (G6PDH), phosphite dehydrogenase (PDH) or a combination of one or more thereof. The enzyme-coupled regeneration system further comprises the substrate for the enzyme reaction that simultaneously generates the ADH-cofactor that assists the conversion of undecavertol to undecavertone or the conversion of undecavertone to undecavertol. For example, the enzyme-coupled regeneration system further comprises the substrate for the enzyme reaction that simultaneously generates the oxidised ADH-cofactor (e.g. NAD⁺ and/or NADP⁺) or the reduced ADH-cofactor (e.g. NADH and/or NADPH).

For example, the substrate of NADH oxidase and NADPH oxidase may be oxygen, which is converted to superoxide (which may form water or hydrogen peroxide) as a result of the enzyme reaction of NADH oxidase and NADPH oxidase. Iron (III) porphyrine mimics NAD(P)H oxidase and thus the substrate for iron (III) porphyrine is oxygen, which is converted to superoxide (which may form water). For example, the substrate of laccase may be oxygen, which may be converted to water. For example, the substrate of lactate dehydrogenase may be pyruvate, which may be converted to lactate, for example as it converts NADH and/or NADPH to NAD⁺ and/or NADP⁺. For example, the substrate of glutamate dehydrogenase may be ketoglutarate and/or ketoadipate, which may be converted to glutamate and/or aminoadipate respectively, for example as it converts NADH and/or NADPH to NAD⁺ and/or NADP⁺. These enzyme-coupled regeneration systems may be particularly useful to regenerate the ADH-cofactor used to convert undecavertol to undecavertone.

For example, the substrate of formiate dehydrogenase (FDH) may be formiate, which is converted to carbon dioxide as a result of the enzyme reaction of FDH. For example, the substrate of glucose dehydrogenase (GDH) may be glucose, which is converted to gluconolactone or gluconate. For example, the substrate of glucose-6-phosphate dehydrogenase (G6PDH) may be glucose-6-phosphate, which is converted to 6-phosphogluconolactone or 6-phosphogluconate. For example, the substrate of phosphite dehydrogenase (PDH) may be phosphite, which is converted to phosphate. These enzyme-coupled regeneration systems may be particularly useful to regenerate the ADH-cofactor used to convert undecavertone to undecavertol.

The enantiomeric mixture of undecavertol or the undecavertone may, for example, be contacted with the ADH and/or ADH-cofactor and/or ADH-cofactor regeneration system themselves or may, for example, be contacted with an expression system capable of expressing the ADH and/or ADH-cofactor and/or ADH-cofactor regeneration system under conditions suitable for expressing the ADH and/or ADH-cofactor and/or ADH-cofactor regeneration system. For example, the enantiomeric mixture of undecavertol or the undecavertone may be contacted with a microorganism or cell that expresses the ADH and/or ADH-cofactor (more often referred to as whole-cell catalysis).

Alternatively, the enantiomeric mixture of undecavertol or the undecavertone may, be contacted with any composition containing ADH and/or ADH-cofactor and/or ADH-cofactor regeneration system, for example, may be contacted with fermentation broth, homogenized broth, cell-free extract or purified ADH and/or ADH-cofactor and/or ADH-cofactor regeneration system. For example, fermentation broth, homogenized broth, cell-free extract or purified ADH and/or ADH-cofactor and/or ADH-cofactor regeneration system may be in solution or may, for example be in freeze-dried or spray-dried form.

The contacting of the enantiomeric mixture of undecavertol or the undecavertone, ADH, ADH-cofactor and optional ADH-cofactor regeneration system may, for example, take place under conditions suitable to convert one undecavertol enantiomer to undecavertone or to convert undecavertone to one enantiomer of undecavertol. The contacting of the enantiomeric mixture of undecavertol or the undecavertone, ADH, ADH-cofactor and optional ADH-cofactor regeneration system may, for example, take place by mixing each component together. The contacting of the enantiomeric mixture of undecavertol or the undecavertone, ADH, ADH-cofactor and optional ADH-cofactor regeneration system may, for example, take place in an aqueous reaction medium, for example in a buffer, for example a phosphate buffer such as a potassium phosphate buffer or a Tris (tris(hydroxymethyl)aminomethane) buffer. Alternatively, the contacting step may take place in an organic reaction medium or an organic/aqueous biphasic reaction medium. For example, where a substrate-coupled cofactor regeneration system is used, the contacting step may take place in an organic reaction medium (e.g. dimethylsulfoxide (DMSO), or tetrahydrofuran (THF)) or an organic/aqueous biphasic reaction medium.

The enantiomeric mixture of undecavertol or the undecavertone, ADH and ADH-cofactor may be added in any amounts and in any proportion suitable to obtain the desired result in a desired period of time.

The concentration of the ADH-substrate (e.g. the enantiomeric mixture of undecavertol or undecavertone) may, for example be equal to or greater than about 10 mM. For example, the concentration of the ADH-substrate may be equal to or greater than about 100 mM or equal to or greater than about 500 mM or equal to or greater than about 1 M. The concentration of the ADH-substrate may, for example, be equal to or less than about 5 M, for example equal to or less than about 4 M, for example equal to or less than about 3 M, for example equal to or less than about 2 M, for example equal to or less than about 1 M.

Enzyme activity may be measured in “units” or “U”, where 1 U corresponds to the amount of enzyme that converts 1 micromol of substrate per minute. In certain embodiments, the enzyme activity of the cofactor regeneration system may be greater than the enzyme activity of the ADH so as to not limit the ADH through cofactor shortage. For example, the enzyme activity of the cofactor regeneration system is at least about 2 times or at least about 4 times or at least about 5 times or at least about 6 times or at least about 8 times or at least about 10 times greater than the enzyme activity of the ADH. For example, the enzyme activity of the cofactor regeneration system may be up to about 20 times or up to about 18 times or up to about 16 times or up to about 15 times greater than the enzyme activity of the ADH.

The ratio of substrate (e.g. enantiomeric mixture of undecavertol) to enzyme activity of ADH (U/mmol) may be varied depending on the desired reaction time and stability of the enzyme. In certain embodiments, the ratio of substrate to enzyme activity of ADH is equal to or greater than about 0.1 U/mmol, for example equal to or greater than about 0.2 U/mmol, for example equal to or greater than about 0.3 U/mmol, for example equal to or greater than about 0.4 U/mmol, for example equal to or greater than about 0.5 U/mmol. For example, the ratio of substrate to enzyme activity of ADH is equal to or less than about 20 U/mmol, for example equal to or less than about 15 U/mmol, for example equal to or less than about 10 U/mmol.

The concentration of the cofactor may, for example, be equal to or greater than about 0.001 mol %. For example, the concentration of the cofactor may be equal to or greater than about 0.005 mol %, for example equal to or greater than about 0.01 mol %. For example, the concentration of the cofactor may be equal to or less than about 10 mol %, for example equal to or less than about 5 mol %.

The contacting of the enantiomeric mixture of undecavertol or the undecavertone, ADH, ADH-cofactor and optional ADH-cofactor regeneration system may, for example, take place for a period of time required to obtain a desired result. For example, the contacting of the enantiomeric mixture of undecavertol or the undecavertone, ADH, ADH-cofactor and optional ADH-cofactor regeneration system may take place for a period of time ranging from about 30 minutes to about 3 days. For example, the contacting of the enantiomeric mixture of undecavertol or the undecavertone, ADH, ADH-cofactor and optional ADH-cofactor regeneration system may take place for a period of time ranging from about 30 minutes to about 2 days or from about 30 minutes to about 24 hours or from about 1 hour to about 18 hours or from about 2 hours to about 12 hours or from about 6 hours to about 12 hours or from about 6 hours to about 10 hours.

The contacting of the enantiomeric mixture of undecavertol or the undecavertone, ADH, ADH-cofactor and optional ADH-cofactor regeneration system may, for example, take place at a temperature lower than the denaturing temperature of the enzymes used and/or a temperature higher than the melting point of the solvent or solvent mixture. A person skilled in the art will be able to select a suitable temperature depending on the particular enzymes used. For example, the contacting of the enantiomeric mixture of undecavertol or the undecavertone, ADH, ADH-cofactor and optional ADH-cofactor regeneration system may take place at a temperature ranging from about 20° C. to about 40° C. For example, the contacting may take place at a temperature ranging from about 22° C. to about 38° C. or from about 24° C. to about 36° C. or from about 25° C. to about 35° C. or from about 28° C. to about 32° C., for example about 30° C.

The components may be stirred or shaken, for example at 100 rpm to about 500 rpm or from about 150 rpm to about 450 rpm or from about 200 rpm to about 400 rpm or from about 250 rpm to about 350 rpm.

The method provided herein may further comprise purification of the undecavertol and/or separation from any undecavertone. For example, the undecavertol may be purified by solvent extraction (e.g. using methyl-tert-butyl-ether, MTBE) followed by distillation.

There is further provided herein enantiomeric mixtures obtained by and/or obtainable by the methods provided herein. The enantiomeric mixture of undecavertol may have an enantiomeric excess equal to or greater than about 94%. For example, the enantiomeric mixture may have an enantiomeric excess equal to or greater than about 95% or equal to or greater than about 96% or equal to or greater than about 97% or equal to or greater than about 98% or equal to or greater than about 99%. In certain embodiments, the enantiomeric mixture may have an enantiomeric excess equal to or less than about 100% or equal to or less than about 99.5% or equal to or less than about 99%. The enantiomeric mixture may have an enantiomeric excess of either the (R)-enantiomer or the (S)-enantiomer. In certain embodiments, the enantiomeric mixture has an enantiomeric excess of (R)-enantiomer.

There is further provided herein the use of the enantiomeric mixtures of undecavertol obtained by and/or obtainable by the methods provided herein as a fragrance. Thus, there is also provided herein a fragrance composition comprising an enantiomeric mixture of undecavertol obtained by and/or obtainable by the methods provided herein.

The undecavertol obtained by and/or obtainable by the methods provided herein is a highly blooming fragrance ingredient. In particular a mixture having an enantiomeric excess equal to or greater than about 94% of (R)-undecavertol (including mixtures having an ee of equal or greater than about 95%, 96%, 97%, 98%, 99%, 99.5%, 99.6%, 99.7%, 99.8%, 99.9% of (R)-undecavertol) is a highly blooming fragrance ingredient.

A fragrance “bloom” refers to the short term impact of an ingredient at a certain distance from the fragrance source. Short term means a few seconds to a few minutes after an external action has been applied to the fragrance ingredient itself. Such an action (or event) can be multiple in nature. Opening a fragrance flask, spraying a fragrance solution in the air or on the skin, contacting a perfumed product with a surface or with water, and in particular diluting a perfumed product with water, are typical actions capable of inducing a bloom. Perfumes and individual fragrance ingredients can be classified as low blooming to high blooming perfume/fragrance ingredients. A high bloom is highly desired by consumers in the current market place.

Although “bloom” is typically a time-dependent dynamic performance attribute of a fragrance it is measured after a certain time, but not later than 30 minutes, preferably 15 to 20 minutes, after the action has taken place. Typically, the assessment is performed in a closed volume of air, for example in a non-ventilated booth. Typically a panelist performs the assessment by smelling a certain volume of air (e.g. one or two breaths) in the booth through a small window which is open only during the assessment. Typically, the window is located between 0.5 meters and 2 meters from the source, preferably between 0.8 meters and 1.5 meters, for example 1.3 meters from the source. The exact geometry of the experimental set-up is not critical, but it must be reproducible from one assessment to the other.

By “fragrance composition” is meant any composition comprising an enantiomeric mixture of undecavertol obtained by and/or obtainable by the methods provided herein and a base material.

As used herein, the “base material” includes all known fragrance ingredients selected from the extensive range of natural products, and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in fragrance compositions, for example, carrier materials, diluents, and other auxiliary agents commonly used in the art.

Fragrance ingredients known to the art are readily available commercially from the major fragrance manufacturers. Non-limiting examples of such ingredients include:
essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, vetiver oil, wormwood oil, lavender oil and/or ylang-ylang oil;
alcohols, e.g. cinnamic alcohol ((E)-3-phenylprop-2-en-1-ol); cis-3-hexenol ((Z)-hex-3-en-1-ol); citronellol (3,7-dimethyloct-6-en-1-ol); dihydro myrcenol (2,6-dimethyloct-7-en-2-ol); Ebanol™ ((E)-3-methyl-5-(2,2,3-trimethylcyclopent-3-en-1-yl) pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-ol); Sandalore™ (3-methyl-5-(2,2,3-trimethylcyclopent-3-en-1-yl)pentan-2-ol); terpineol (2-(4-methylcyclohex-3-en-1-yl)propan-2-ol); or Timberol™ (1-(2,2,6-trimethylcyclohexyl)hexan-3-ol); 2,4,7-trimethylocta-2,6-dien-1-ol, and/or [1-methyl-2(5-methylhex-4-en-2-yl)cyclopropyl]-methanol;
aldehydes and ketones, e.g. anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl)ethanone); Hydroxycitronellal (7-hydroxy-3,7-dimethyloctanal); Iso E Super® (1-(2,3,8,8-tetramethyl-1,2,3,4,5,6,7,8-octahydronaphthalen-2-yl)ethanone); Isoraldeine® ((E)-3-methyl-4-(2,6,6-trimethylcyclohex-2-en-1-yl)but-3-en-2-one); 3-(4-isobutyl-2-methylphenyl)propanal; (E)-9-hydroxy-5,9-dimethyldec-4-enal; maltol; methyl cedryl ketone; methylionone; verbenone; and/or vanillin;
ether and acetals, e.g. Ambrox® (3a,6,6,9a-tetramethyl-2,4,5,5a,7,8,9,9b-octahydro-1H-benzo[e][1]benzofuran); geranyl methyl ether ((2E)-1-methoxy-3,7-dimethylocta-2,6-diene); rose oxide (4-methyl-2-(2-methylprop-1-en-1-yl)tetrahydro-2H-pyran); and/or Spirambrene® (2′,2′,3,7,7-pentamethylspiro[bicyclo[4.1.0]heptane-2,5′-[1,3]dioxane]);
macrocycles, e.g. Ambrettolide ((Z)-oxacycloheptadec-10-en-2-one); ethylene brassylate (1,4-dioxacycloheptadecane-5,17-dione); and/or Exaltolide® (16-oxacyclohexadecan-1-one); and
heterocycles, e.g. isobutylquinoline (2-isobutylquinoline).

As used herein, “carrier material” means a material which is practically neutral from a odorant point of view, i.e. a material that does not significantly alter the organoleptic properties of odorants.

By “diluents” is meant any diluent conventionally used in conjunction with odorants, such as diethyl phthalate (DEP), dipropylene glycol (DPG), isopropyl myristate (IPM), triethyl citrate (TEC) and alcohol (e.g. ethanol).

The term “auxiliary agent” refers to ingredients that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same, such as anti-oxidant adjuvant. Said anti-oxidant may be selected, for example, from Tinogard® TT (BASF), Tinogard® Q (BASF), Tocopherol (including its isomers, CAS 59-02-9; 364-49-8; 18920-62-2; 121854-78-2), 2,6-bis(1,1-dimethylethyl)-4-methylphenol (BHT, CAS 128-37-0) and related phenols, hydroquinones (CAS 121-31-9).

It might also be an ingredient that provides additional benefits such as imparting colour or texture. It might also be an ingredient that imparts light resistance or chemical stability to one or more ingredients contained in a fragrance composition.

A detailed description of the nature and type of auxiliary agent commonly used in fragrance compositions containing same cannot be exhaustive, but it has to be mentioned that said ingredients are well known to a person skilled in the art.

The present invention is further based, at least in part, on the surprising finding that a specific enantiomer of undecavertol can be stereoselectively synthesised by asymmetric hydrogenation of undecavertone using hydrogen (H₂). Either the (R)-enantiomer or the (S)-enantiomer may be stereoselectively synthesised. However, in certain embodiments, the (R)-enantiomer is synthesised by the asymmetric hydrogenation of undecavertone.

The asymmetric hydrogenation of undecavertone may, for example, be facilitated by a catalyst. The catalyst may, for example, be an inorganic catalyst such as RuCl₂[(S)-xylbinap][(S,S)-dpen] (Dichloro[(S)-2,2′-bis[di(3,5-xylyl)phosphino]-1,1′-binaphthyl] [(S,S)-1,2-diphenylethylenediamine]ruthenium(II)).

The asymmetric hydrogenation of undecavertone may, for example, take place in the presence of one or more further reagents (in addition to the hydrogen) such as, for example, isopropanol, potassium t-butoxide, potassium hydroxide or a combination thereof.

The asymmetric hydrogenation may take place under any conditions suitable for the stereoselective hydrogenation of undecavertone. For example, the asymmetric hydrogenation may take place at a hydrogen pressure ranging from about 10 bar (1000 kPa) to about 100 bar (10,000 kPa), for example from about 25 bar (2500 kPa) to about 75 bar (7500 kPa), for example from about 40 bar (4000 kPa) to about 60 bar (6000 kPa), for example about 50 bar (5000 kPa). For example, the asymmetric hydrogenation may take place at a temperature ranging from about 25° C. to about 75° C., for example from about 30° C. to about 70° C., for example from about 40° C. to about 60° C., for example about 50° C.

The undecavertol may then be purified, which may involve separation from the undecavertone, by means known to the person skilled in the art. For example, the undecavertol may be purified by distillation.

The undecavertone used in the asymmetric hydrogenation may, for example, be made using an enantiomeric mixture of undecavertol and ADH as described herein. Alternatively, the undecavertone may be made by another method, for example by an Oppenauer oxidation method. This method may use an enantiomeric mixture of undecavertol and aluminium isopropoxide catalyst in excess acetone, benzaldehyde or furfural. Any suitable conditions may be used. For example, the oxidation may take place at a temperature ranging from about 50° C. to about 90° C., for example from about 60° C. to about 80° C., for example about 70° C.

### Example 1 (Acetone Cofactor Regeneration)

A 5 mL parallel shaking reactor was charged with 2 mL of alcohol dehydrogenase solution (12.5 g L⁻¹ KRED-P1-B10 in KPi buffer (100 mM, pH 7.5) obtained from Codexis Inc., Redwood City, (USA)). 0.25 mL of NADP⁺ solution (15.8 g L⁻¹ in KPi buffer (100 mM, pH 7.5)), 2.25 mL KPi buffer (100 mM, pH 7.5) and 0.5 mL rac-trans-undecavertol solution (0.5 M in acetone) were added. The reaction was shaken at 30° C. and 250 rpm. After 8 hours, a sample of the reaction was taken, extracted with methyl-tert-butyl-ether (MTBE) and the organic phase analyzed via gas chromatography.
Conversion: 52.2%
Enantiomeric excess: 99.4% (R)-trans-undecavertol

### Example 2 (NOX Cofactor Regeneration)

A 5 mL parallel shaking reactor was charged with 0.524 mL of alcohol dehydrogenase solution (50.0 g L⁻¹ ADH-87 obtained from c-LEcta GmbH Leipzig (Germany)) in Tris buffer (100 mM, pH 8.0). 0.05 mL of NADP⁺ solution (78.7 g L⁻¹ in Tris buffer (100 mM, pH 8.0)), 0.250 mL Tris buffer (100 mM, pH 8.0), 0.176 mL NAD(P)H oxidase cell-free extract from Streptococcus mutans (49 g L⁻¹ protein content) obtained from InnoSyn B.V., Geleen (The Netherlands) and 4.0 mL rac-trans-undecavertol were added. The reaction was shaken at 30° C. and 350 rpm. After 24 hours, a sample of the organic layer was taken, diluted with MTBE and analyzed via gas chromatography.
Conversion: 54.2%
Enantiomeric excess: 99.2% (R)-trans-undecavertol

### Example 3 (Acetone Cofactor Regeneration)

A 1.5 mL capped tube was charged with 0.85 mL of alcohol dehydrogenase solution (20 g L⁻¹ KRED-P3-G09 (obtained from Codexis, Inc., Redwood City, USA) in KPi buffer (100 mM, pH 7.5)). 0.05 mL of NADP⁺ solution (39.4 g L⁻¹ in KPi buffer (100 mM, pH 7.5)) and 0.1 mL of substrate solution (21 g L⁻¹ rac-trans-undecavertol in acetone) were added. The reaction was shaken at 30° C. and 600 rpm. After 24 hours, the reaction mixture was extracted with MTBE and analyzed via gas chromatography.
Enantiomeric excess: 98.8% (S)-trans-undecavertol

### Example 4 (Isopropanol Cofactor Regeneration)

A 1.5 mL capped tube was charged with 0.85 mL of alcohol dehydrogenase solution (10 g L⁻¹ KRED-P3-G09 (obtained from Codexis, Inc., Redwood City, (USA)) in KPi buffer (100 mM, pH 7.5)). 0.05 mL of NADP⁺ solution (39.4 g L⁻¹ in KPi buffer (100 mM, pH 7.5)) and 0.1 mL of substrate solution (84.1 g L⁻¹ undecavertone in isopropanol) were added. The reaction was shaken at 30° C. and 600 rpm. After 24 hours, the reaction mixture was extracted with MTBE and analyzed via gas chromatography.
Enantiomeric excess: 99.2% (R)-trans-undecavertol

### Example 5 (Asymmetric Hydrogenation of Undecavertone)

Under air atmosphere, RuCl₂[(S)-xylbinap][(S,S)-dpen] (10 mg, 0.01 wt %) was added to a solution of (E)-4-methyldec-3-en-5-one (90 g, 535 mmol), potassium t-butoxide (0.6 g, 5.35 mmol) in i-PrOH (90 g) in a 1 L autoclave vessel and sealed. Whilst stirring, the autoclave was flushed three times with N₂, then three times with H₂. The H₂-pressure was set to 47 bar, heated to 50° C. and stirred. After consumption of the starting material the heating of the autoclave and the H₂-Flow were stopped. Once cooled, the pressure was released and the autoclave was flushed three times with N₂. The solvent was removed from the crude yellow reaction mixture. The crude material was dissolved in MTBE (100 mL), transferred to a 500 mL separating funnel and washed with H₂O (100 mL). The aqueous layer was re-extracted with MTBE (100 mL) and the combined organic layers were washed with brine, dried over MgSO₄ and filtered. Solvent removal provided a yellow liquid, which, after distillation (Sulzer packed column) gave (R)-undecavertol (81.5 g, 83.5% yield, 96.4% ee) as a colorless liquid.

The foregoing broadly describes certain embodiments of the present invention without limitation. Variations and modifications as will be readily apparent to those skilled in the art are intended to be within the scope of the present invention as defined in and by the appended claims.

### Example 6 (NOX Cofactor Regeneration)

A 1 L laboratory reactor was charged with 90 mL Tris buffer (100 mM, pH 8.0), 2.62 g ADH-87 (obtained from c-LEcta GmbH Leipzig (Germany)), 0.079 g NADP⁺ disodium salt, 10 mL NAD (P) H oxidase cell-free extract from Streptococcus mutans (obtained from InnoSyn B.V., Geleen (The Netherlands)) and 340.6 g rac-trans-undecavertol. The reaction was sparged with oxygen (150 mL min⁻¹) and stirred at 30° C. and 500 rpm. Samples of the organic layer was taken, diluted with MTBE and analyzed via gas chromatography to follow the progress of the reaction. The reaction was stopped after 24 h and the layers were separated. The aqueous layer was reextracted with MTBE and the organic solvent from the combined organic layers was removed in vacuo. The product was purified via distillation.
Conversion: 51.2%
Enantiomeric excess: 97.2% (R)-trans-undecavertol

1. An enantiomeric mixture of undecavertol, the mixture having an enantiomeric excess equal to or greater than about 94%.

2. The enantiomeric mixture of undecavertol of claim 1, wherein the mixture is stereoselectively synthesized to have an enantiomeric excess equal to or greater than about 94%.

3. The enantiomeric mixture of undecavertol of claim 1, the mixture having an enantiomeric excess equal to or greater than about 95%.

4. The enantiomeric mixture of undecavertol of claim 1, the mixture having an enantiomeric excess equal to or greater than about 96%.

5. The enantiomeric mixture of undecavertol of claim 1, the mixture having an enantiomeric excess equal to or greater than about 97%.

6. The enantiomeric mixture of undecavertol of claim 1, the mixture having an enantiomeric excess equal to or greater than about 98%.

7. The enantiomeric mixture of undecavertol of claim 1, the mixture having an enantiomeric excess equal to or greater than about 99%.

8. The enantiomeric mixture of undecavertol of claim 1, wherein the mixture has an enantiomeric excess of the (R)-enantiomer.

9. The enantiomeric mixture of undecavertol of claim 3, wherein the mixture has an enantiomeric excess of the (R)-enantiomer.

10. The enantiomeric mixture of undecavertol of claim 4, wherein the mixture has an enantiomeric excess of the (R)-enantiomer.

11. The enantiomeric mixture of undecavertol of claim 5, wherein the mixture has an enantiomeric excess of the (R)-enantiomer.

12. The enantiomeric mixture of undecavertol of claim 6, wherein the mixture has an enantiomeric excess of the (R)-enantiomer.

13. The enantiomeric mixture of undecavertol of claim 7, wherein the mixture has an enantiomeric excess of the (R)-enantiomer.

14. A fragrance composition comprising a base material and the enantiomeric mixture of undecavertol according to claim 1.

15. A method of utilizing the enantiomeric mixture of undecavertol according to claim 1 as a fragrance, wherein the method comprises mixing the enantiomeric mixture of undecavertol with a base material.
